(19) United States
(12) Patent Application Publication
(10) Pub. No.: US 2020/0323457 A1
(43) Pub. Date: Oct. 15, 2020

(54) HEARTBEAT ANALYZER

(71) Applicant: NIHON KOHDEN CORPORATION, Tokyo (JP)

(72) Inventors: Masafumi NAGASAWA, Tokorozawa-shi (JP); Kazuhiro SEKIKAWA, Tokorozawa-shi (JP); Kei KIMURA, Tokorozawa-shi (JP)

(21) Appl. No.: 16/830,846

(22) Filed: Mar. 26, 2020

(30) Foreign Application Priority Data
Apr. 11, 2019 (JP) 2019-075584

(51) Int. Cl. A61B 5/0432; A61B 5/0464; A61N 1/365; A61N 1/37

(52) U.S. Cl. CPC A61B 5/0432; A61N 1/3706; A61N 1/36592; A61B 5/0464

(57) ABSTRACT

A heartbeat analyzer analyzes heartbeats of a living body to which a pacing device for outputting pacing pulses to cause a heart to beat is placed. The heartbeat analyzer includes: a heartbeat detecting section that detects the heartbeats; a pulse detecting section that detects outputs of the pacing pulses; a heartbeat determining section that determines whether the heartbeats are paced beats due to the pacing pulses or not, based on detection timings of the pacing pulses; a tachycardia determining section that, in a case where the paced beats are in a number that is equal to or larger than a predetermined heart rate, and are contained in the heartbeats at a predetermined ratio or more, determines that pacing tachycardia caused by the pacing pulses occurs in the living body; and a notifying section that notifies of the occurrence of the pacing tachycardia.

### CROSS-REFERENCE TO RELATED APPLICATIONS

This application is based upon and claims the benefit of priority from prior Japanese patent application No. 2019-075584, filed on Apr. 11, 2019, the entire contents of which are incorporated herein by reference.

### TECHNICAL FIELD

The presently disclosed subject matter relates to a heartbeat analyzer, and particularly to an apparatus for, based on electrical signals of a living body to which a pacing device is placed, analyzing heartbeat of the living body.

### BACKGROUND ART

A related-art heartbeat analyzer is available that receives electrical signals from electrodes placed on a living body, and that analyzes heartbeats of the living body based on the electrical signals. When the heartbeat analyzer calculates the heart rate of the living body based on the electrical signals, it is possible to determine an arrhythmia such as tachycardia. It is often that an arrhythmia must be rapidly treated, and therefore it is requested to correctly determine the occurrence thereof.

As a technique for correctly determining an arrhythmia, for example, JP-A-2014-150826 discloses an electrocardiogram analyzer that prevents a failure in which an arrhythmia is determined as a reference waveform. In the electrocardiogram analyzer, an electrocardiogram waveform of the living body per heartbeat is compared with the reference waveform to determine whether an arrhythmia occurs or not, and therefore it is possible to correctly determine an arrhythmia.

In the case where a pacing device such as a pacemaker that outputs pacing pulses to cause the heart to beat is placed to the living body, however, the electrocardiogram analyzer disclosed in JP-A-2014-150826 does not determine whether tachycardia that appears in the electrocardiogram is pacing tachycardia triggered by pacing pulses or not. Therefore, there remains room for improvement in discrimination between pacing tachycardia and spontaneous tachycardia of the living body itself.

The presently disclosed subject matter has been conducted in order to solve the problem of the related art. It is an object of the presently disclosed subject matter to provide a heartbeat analyzer that distinguishes more clearly pacing tachycardia.

### SUMMARY OF INVENTION

According to an aspect of the presently disclosed subject matter, there is provided a heartbeat analyzer that is configured to analyze heartbeats of a living body to which a pacing device for outputting pacing pulses to cause a heart to beat is placed, based on electrical signals of the living body, the heartbeat analyzer comprising: a heartbeat detecting section that is configured to detect the heartbeats of the living body based on the electrical signals; a pulse detecting section that is configured to detect outputs of the pacing pulses based on the electrical signals; a heartbeat determining section that is configured to determine whether the heartbeats detected by the heartbeat detecting section are paced beats due to the pacing pulses detected by the pulse detecting section or not, based on detection timings of the pacing pulses; a tachycardia determining section that, in a case where the paced beats determined by the heartbeat determining section are in a number that is equal to or larger than a predetermined heart rate, and are contained in the heartbeats at a predetermined ratio or more, is configured to determine that pacing tachycardia caused by the pacing pulses occurs in the living body; and a notifying section that is configured to notify of the occurrence of the pacing tachycardia based on a result of the determination of the tachycardia determining section.

### BRIEF DESCRIPTION OF DRAWINGS

FIG. 1 is a block diagram illustrating the configuration of a heartbeat analyzer of Embodiment 1 of the presently disclosed subject matter.

FIG. 2 is a flowchart illustrating the operation of Embodiment 1.

FIG. 3 illustrates the manner of a heartbeat waveform that is adjusted by pacing pulses.

FIG. 4 is a block diagram illustrating main portions of a heartbeat analyzer of Embodiment 2.

FIG. 5 is a flowchart illustrating the operation of Embodiment 2.

FIG. 6 is a block diagram illustrating main portions of a heartbeat analyzer of Embodiment 3.

FIG. 7 is a flowchart illustrating the operation of Embodiment 3.

FIG. 8 is a flowchart illustrating the operation of Embodiment 4.

FIG. 9 is a flowchart illustrating the operation of Embodiment 5.

### DESCRIPTION OF EMBODIMENTS

Hereinafter, embodiments of the presently disclosed subject matter will be described with reference to the accompanying drawings.

### Embodiment 1

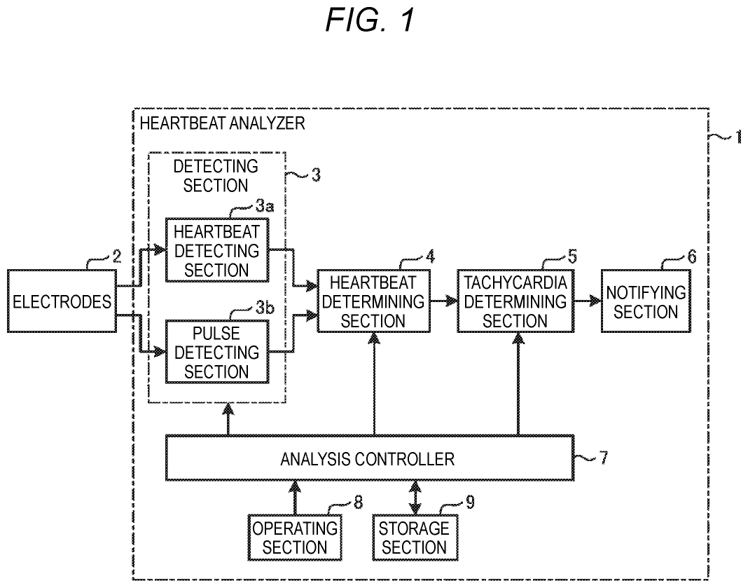

FIG. 1 illustrates the configuration of a heartbeat analyzer 1 of Embodiment 1 of the presently disclosed subject matter. The heartbeat analyzer 1 may include a detecting section 3 that is connected to electrodes 2, and a heartbeat determining section 4, a tachycardia determining section 5, and a notifying section 6 are sequentially connected to the detecting section 3. An analysis controller 7 is connected to the detecting section 3, the heartbeat determining section 4, and the tachycardia determining section 5. An operating section 8 and a storage section 9 are connected to the analysis controller 7.

The electrodes 2 are placed on the living body to which a pacing device is placed, and receive electrical signals from the living body. Here, the pacing device sequentially outputs pacing pulses (one kind of action pulses) toward the heart of the living body to cause the heart to beat. Therefore, not only electrical signals indicating the operation of the heart, but also pacing pulses that are output from the pacing device are input to the electrodes 2.

Examples of the pacing device are a pacemaker and the like.

The detecting section 3 may include a heartbeat detecting section 3a and pulse detecting section 3b that are connected to the electrodes 2.

The heartbeat detecting section 3a detects the heartbeats of the living body based on the electrical signals that are input to the electrodes 2. For example, the heartbeat detecting section 3a may compare waveforms of the electrical signals that are input to the electrodes 2, with a reference heartbeat waveform that is previously stored, and detect the heartbeats of the living body based on the similarity therebetween. Here, the heartbeat detecting section 3a detects not only spontaneous beats that are generated by causing the heart to beat, by the living body itself, but also paced beats that are generated by beating of the heart of the living body that is cased by pacing pulses.

The pulse detecting section 3b detects outputs of the pacing pulses from the pacing device based on the electrical signals that are input to the electrodes 2. For example, the pulse detecting section 3b may detect, as the pacing pulses, electrical signals that are input to the electrodes 2 as a differential signal.

The heartbeat determining section 4 is connected to the heartbeat detecting section 3a and the pulse detecting section 3b, and determines whether the heartbeats detected by the heartbeat detecting section 3a are paced beats due to the pacing pulses or not, based on the detection timings of the pacing pulses that are detected by the pulse detecting section 3b.

In the case where the paced beats that are determined by the heartbeat determining section 4 are in a number that is equal to or larger than a predetermined heart rate, and contained in the heartbeats detected by the heartbeat detecting section 3a, at the predetermined ratio or more, the tachycardia determining section 5 determines that pacing tachycardia in which the heartbeats of the living body are caused by the pacing pulses to be quickened occurs.

Here, the heart rate of the paced beats indicates the number of the paced beats per minute, and can be obtained based on, for example, the R-R interval of the paced beats, or the number of the paced beats in a constant time period. The ratio of the paced beats indicates the generation rate of the paced beats, and, for example, may be obtained from the generated number of paced beats occupying in recent several or several tens of heartbeats, or heartbeats in a constant time period.

The notifying section 6 notifies of the occurrence of pacing tachycardia based on the result of the determination of the tachycardia determining section 5. For example, the notifying section 6 may be configured by a display device such as a liquid crystal display, a printer, a speaker, or a warning light.

The analysis controller 7 controls the components of the heartbeat analyzer 1 based on instructions that are input by the user through the operating section 8.

The operating section 8 is used for inputting instructions given by the user, and may be formed by a keyboard, a mouse, a track ball, a touch panel, or the like.

The storage section 9 stores operation programs and the like, and may use a recording medium such as a hard disk, a flexible disk, an MO, an MT, a RAM, a CD-ROM, a DVD-ROM, an SD card, a CF card, or a USB memory, or a server or the like may be employed.

The heartbeat determining section 4, the tachycardia determining section 5, and the analysis controller 7 are configured by a CPU and operation programs that cause the CPU to execute various processes, but alternatively may be configured by digital circuits.

The heartbeat analyzer 1 may be used in an apparatus for analyzing heartbeats, and, for example, used while being incorporated in an electrocardiogram monitor, a diagnostic electrocardiograph, a Holter electrocardiograph, an exercise electrocardiogram measurement apparatus, a cardiac catheter polygraph, a defibrillator, a pacemaker, an implantable cardioverter defibrillator (ICD), a telemeter, or the like.

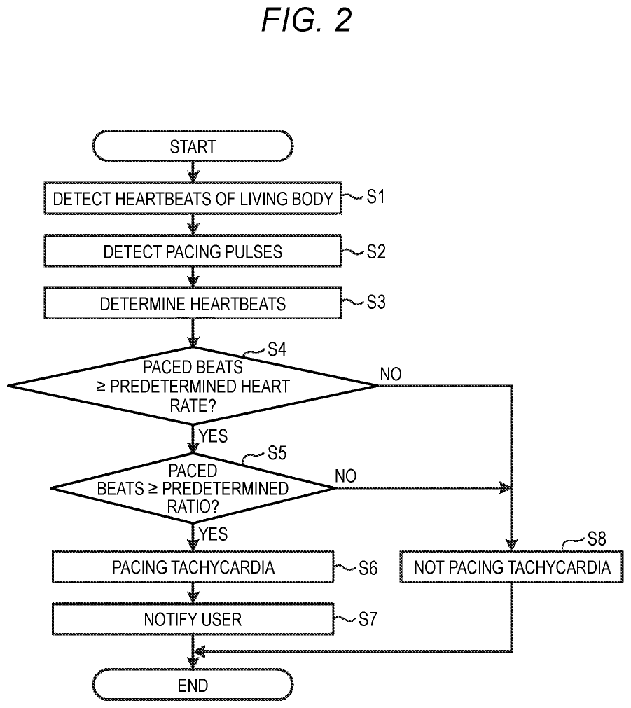

Next, the operation of Embodiment 1 will be described with reference to the flowchart of FIG. 2.

First, the electrodes 2 illustrated in FIG. 1 are placed on the surface of the living body, and electrical signals supplied from the living body are output from the electrodes 2 to the heartbeat detecting section 3a and the pulse detecting section 3b. Here, the pacing device that is not illustrated is placed to the living body, and outputs pacing pulses for causing the heart to beat, toward the heart.

When the electrical signals from the living body are input from the electrodes 2 to the heartbeat detecting section 3a, the heartbeat detecting section 3a detects in step S1 the heartbeats of the living body based on the electrical signals thereof. For example, the heartbeat detecting section 3a compares the electrical signals from the living bod with a reference waveform of the heartbeats that is previously stored, and detects the QRS wave contained in the electrical signals from the living body, i.e., a waveform indicating the ventricular activation, as the heartbeats. In this way, the heartbeat detecting section 3a can detect the heartbeats that contain spontaneous beats and paced beats.

When the electrical signals from the living body are input from the electrodes 2 to the pulse detecting section 3b, moreover, the pulse detecting section 3b detects in step S2 the outputs of the pacing pulses from the pacing device, based on the electrical signals. For example, the pulse detecting section 3b can detect the electrical signals that are input, as a differential signal, from the plurality of electrodes 2 that are placed at different positions, as the pacing pulses.

The detection timings of the heartbeats detected by the heartbeat detecting section 3a, and those of the pacing pulses detected by the pulse detecting section 3b are output to the heartbeat determining section 4. In step S3, then, the heartbeat determining section 4 determines which ones of the paced beats and the spontaneous beats are the heartbeats detected by the heartbeat detecting section 3a, based on the detection timings of the pacing pulses detected by the pulse detecting section 3b.

For example, the heartbeat determining section 4 may determine that a heartbeat that is detected within a predetermined time period from the detection timing of a pacing pulse is a paced beat, and a heartbeat that is detected after the predetermined time period from the detection timing of a pacing pulse is a spontaneous beat. The determination result is output from the heartbeat determining section 4 to the tachycardia determining section 5.

When the result of the determination of the heartbeat is input from the heartbeat determining section 4 to the tachycardia determining section 5, the tachycardia determining section 5 determines in step S4 whether the paced beats that are determined by the heartbeat determining section 4 have a heart rate that is equal to or larger than a predetermined heart rate or not. For example, the tachycardia determining section 5 may calculate the mean or median value of heart rates with using a part or all of paced beats contained in recent several or several tens of beats, or in a constant time period, and determine whether the value is equal to or larger than the predetermined heart rate or not. Here, the predetermined heart rate is preferably set between 15 bpm and 300 bpm, and for example may be set to 100 bpm. Alternatively, the tachycardia determining section 5 may set the predetermined heart rate based on a heart rate that is previously set in order to detect spontaneous tachycardia of the living body itself such as ventricular tachycardia (VT), supraventricular tachycardia (SVT), or overall tachycardia (TACHY) in an electrocardiogram analysis, alarm function, or the like of an electrocardiogram monitor.

If it is determined that the heart rate of the paced beats is equal to or larger than the predetermined heart rate, the process proceeds to step S5, and the tachycardia determining section 5 determines whether or not the paced beats are contained in the heartbeats at a predetermined ratio or more. In the case where the paced beats occupy one half or more of heartbeats in recent several or several tens of heartbeats or in a constant time period, for example, the tachycardia determining section 5 may determine that the paced beats are contained in the heartbeats at the predetermined ratio or more. In the case where the paced beats are at the largest number in recent several or several tens of heartbeats, or heartbeats in a constant time period, i.e., larger in number than spontaneous beats, the tachycardia determining section 5 may determine that the paced beats are contained in the heartbeats at the predetermined ratio or more.

If it is determined that the paced beats are contained in the heartbeats at the predetermined ratio or more, the process proceeds to step S6, and the tachycardia determining section 5 determines that pacing tachycardia occurs in the living body.

When the tachycardia determining section 5 determines whether pacing tachycardia occurs or not, based on only the heart rate of paced beats, there is a possibility that, even in the case where paced beats occur at a certain large interval across a spontaneous beat, i.e., the occurrence ratio of paced beats is low, for example, it is determined that pacing tachycardia occurs. Therefore, the tachycardia determining section 5 determines pacing tachycardia based on the heart rate of the paced beats, and the ratio of the paced beats, and hence can accurately determine an occurrence of pacing tachycardia.

Then, the tachycardia determining section 5 outputs the determination result to the notifying section 6, and, based on the determination result of the tachycardia determining section 5, the notifying section 6 notifies the user of an occurrence of pacing tachycardia in step S7. For example, the notifying section 6 may display “Tachy [P]” in which [P] indicating pacing tachycardia is added to a display of “Tachy” notifying of spontaneous tachycardia of the living body itself, thereby notifying of an occurrence of pacing tachycardia. The notifying section 6 may display a new symbol indicating pacing tachycardia, to notify of an occurrence of pacing tachycardia.

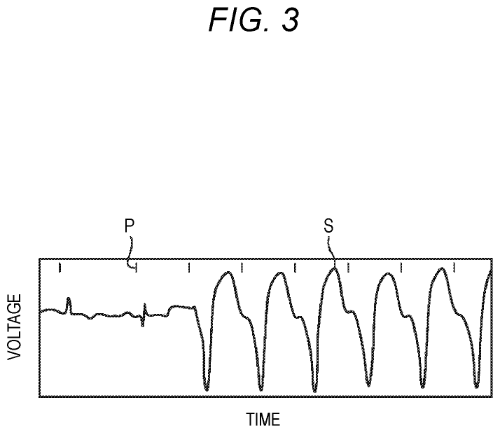

In the case where, as illustrated in FIG. 3, the pacing device causes the output interval of pacing pulses P to be shortened in accordance with, for example, a change of the body temperature of the living body, the user who checks the heartbeat waveform S needs medical knowledge to discriminate between pacing tachycardia and spontaneous tachycardia in which the heartbeat is increased by the living body itself.

When the notifying section 6 notifies the user of an occurrence of pacing tachycardia, therefore, the user can discriminate more clearly an occurrence of pacing tachycardia, and the burden of knowing the heartbeat waveform S that is imposed on the user can be reduced.

If it is determined in step S4 that the number of the paced beats is smaller than the predetermined heart rate, the process proceeds to step S8, and the tachycardia determining section 5 determines that pacing tachycardia does not occur in the living body. Also, if it is determined in step S5 that the paced beats are contained in the heartbeats at a ratio that is smaller than the predetermined ratio, the process proceeds to step S8, and the tachycardia determining section 5 determines that pacing tachycardia does not occur in the living body.

According to the embodiment, in the case where the paced beats that are determined by the heartbeat determining section 4 are in a number that is equal to or larger than the predetermined heart rate, and contained in the heartbeats at the predetermined ratio or more, the arrhythmia determining section 5 determines that pacing tachycardia occurs in the living body, and therefore it is possible to discriminate more clearly pacing tachycardia.

### Embodiment 2

In Embodiment 1 described above, in the case where the paced beats that are determined by the heartbeat determining section 4 occur at a predetermined number of consecutive occurrences or more, the tachycardia determining section 5 preferably determines that the paced beats are contained in the heartbeats at the predetermined ratio or more.

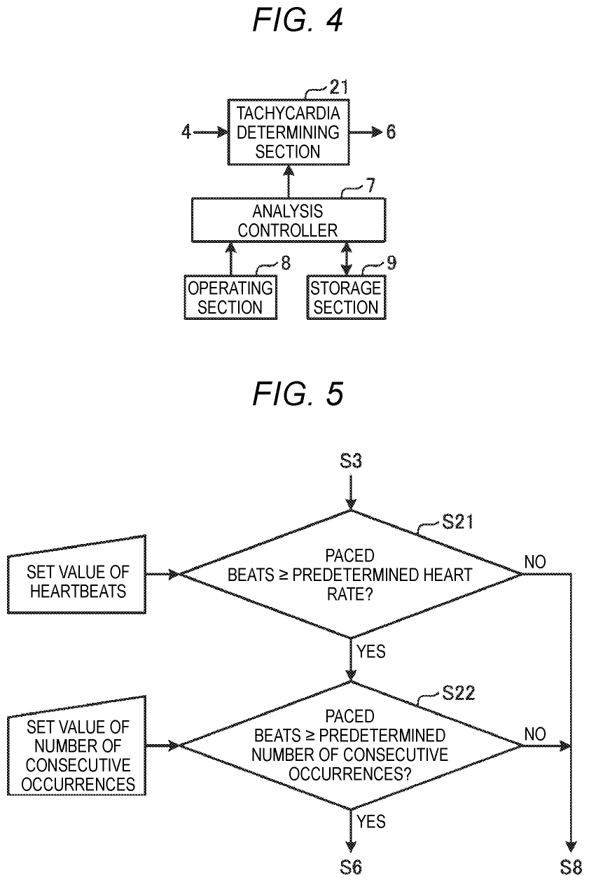

As illustrated in FIG. 4, for example, a tachycardia determining section 21 may be placed in place of the tachycardia determining section 5 in Embodiment 1.

In the case where the paced beats that are determined by the heartbeat determining section 4 are in a number that is equal to or larger than the predetermined heart rate, and contained in the heartbeats at a predetermined number of consecutive occurrences or more, the tachycardia determining section 21 determines that pacing tachycardia occurs in the living body. At this time, the predetermined number of consecutive occurrences may be previously set in the analyzer, or may be set by the user. Alternatively, the predetermined number of consecutive occurrences may be set based on the number of consecutive occurrences that is previously set in order to detect spontaneous tachycardia such as ventricular tachycardia (VT), supraventricular tachycardia (SVT), or overall tachycardia (TACHY) in an electrocardiogram analysis, alarm function, or the like of an electrocardiogram monitor. Here, the predetermined heart rate and the predetermined number of consecutive occurrences may be preferably set by the user through the operating section 8. The number of consecutive occurrences of paced beats indicates the number at which paced beats occur consecutively without being interposed by a spontaneous beat(s).

Next, the operation of Embodiment 2 will be described with reference to the flowchart of FIG. 5.

In the same or similar manner as Embodiment 1, the heartbeat determining section 4 determines in step S3 whether the heartbeats detected by the heartbeat detecting section 3a are paced beats or not, and outputs the result of the determination to the tachycardia determining section 21. Here, the heart rate of paced beats is previously set by the user in the tachycardia determining section 21. In step S21, the tachycardia determining section 21 determines whether or not the number of the paced beats that are determined by the heartbeat determining section 4 is equal to or larger than the heart rate that is set by the user. In the tachycardia determining section 21, the heart rate that can be set by the user may be limited to a range from 15 bpm to 300 bpm.

If it is determined that the number of the paced beats is equal to or larger than the heart rate that is set by the user, the process proceeds to step S22, and the tachycardia determining section 21 determines whether the number of the paced beats is equal to or larger than the predetermined number of consecutive occurrences or not. In the tachycardia determining section 21, the number of consecutive occurrences of paced beats is previously set by the user, or for example to be six or more. The tachycardia determining section 21 performs the determination based on the number of consecutive occurrences that is set by the user. In the tachycardia determining section 21, preferably, the number of consecutive occurrences that can be set by the user is limited to a number which is three or more.

If it is determined that the paced beats occur at the predetermined number of consecutive occurrences or more, the process proceeds to step S6, and the tachycardia determining section 21 determines that pacing tachycardia occurs in the living body.

Since, as described above, the tachycardia determining section 21 determines pacing tachycardia based on the number of consecutive occurrences of paced beats, an occurrence of pacing tachycardia can be determined more accurately than the case where pacing tachycardia is determined based on the ratio of paced beats as in Embodiment 1. Since the tachycardia determining section 21 determines pacing tachycardia based on the heart rate and number of consecutive occurrences that are previously set by the user, determination can be performed in accordance with each living body, and an occurrence of pacing tachycardia can be correctly determined.

By contrast, if it is determined in step S21 that the number of the paced beats is smaller than the heart rate that is set by the user, or if it is determined in step S22 that the number of the paced beats is smaller than the number of consecutive occurrences that is set by the user, the tachycardia determining section 21 determines in step S8 that pacing tachycardia does not occur in the living body, in the same or similar manner as Embodiment 1.

According to the embodiment, in the case where the paced beats that are determined by the heartbeat determining section 4 occur in a number that is equal to or larger than the predetermined heart rate, and equal to or larger than the predetermined number of consecutive occurrences, the tachycardia determining section 21 determines that pacing tachycardia occurs in the living body, and therefore it is possible to determine more accurately an occurrence of pacing tachycardia.

### Embodiment 3

In Embodiments 1 and 2 described above, in the case where paging beats in which their pacing portions due to pacing pulses are identical with each other are contained in the heartbeats at the predetermined ratio or more, the tachycardia determining section preferably determines that pacing tachycardia occurs.

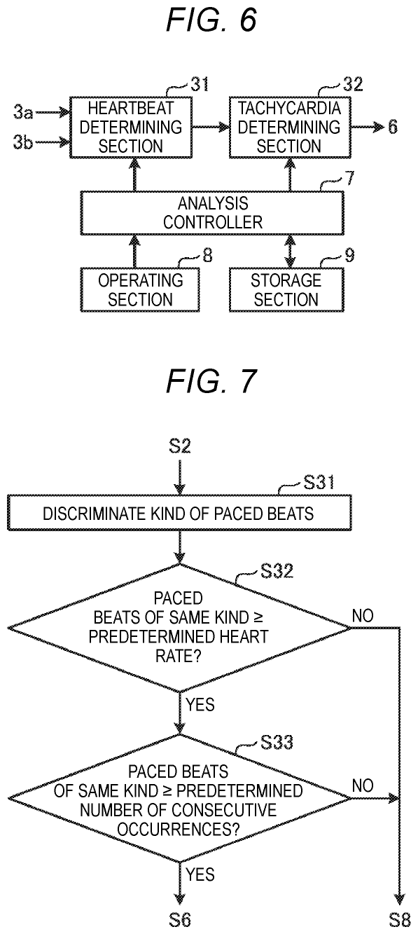

As illustrated in FIG. 6, for example, a heartbeat determining section 31 and a tachycardia determining section 32 may be placed in place of the heartbeat determining section 4 and tachycardia determining section 5 in Embodiment 1.

The heartbeat determining section 31 discriminates the kind of paced beats corresponding to a pacing portion due to pacing pulses, based on the detection timings of the heartbeats that are detected by the heartbeat detecting section 3a, and those of the pacing pulses that are detected by the pulse detecting section 3b. Examples of the kind of paced beats are atrial paced beats, atrial and ventricular paced beats, and ventricular paced beats.

In the case where paced beats of the same kind that is discriminated by the heartbeat determining section 31 are in a number that is equal to or larger than the predetermined number and contained in the heartbeats at the predetermined ratio or more, the tachycardia determining section 32 determines that pacing tachycardia occurs. In the same or similar manner as Embodiment 2, in the case where paced beats of the same kind occur at the predetermined number of consecutive occurrences or more, the tachycardia determining section 32 determines that the paced beats are contained in the heartbeats at the predetermined ratio or more.

Next, the operation of Embodiment 3 will be described with reference to the flowchart of FIG. 7.

In the same or similar manner as Embodiment 1, the pulse detecting section 3b detects in step S2 the output of a pacing pulse from the pacing device, based on the electrical signals supplied from the electrodes 2. In step S31, then, the heartbeat determining section 31 discriminates the kind of the paced beat based on the detection timing of the heartbeat detected by the heartbeat detecting section 3a, and that of the pacing pulse detected by the pulse detecting section 3b.

For example, the heartbeat determining section 31 can discriminate the kind of paced beats based on the time interval between the QRS wave of the paced beat and a pacing pulse that is detected immediately before the QRS wave. Specifically, in the case where the time interval between the pacing pulse and the QRS wave is long, for example, from 0.1 sec. to 0.2 sec., the heartbeat determining section 31 can discriminate the paced beat as that due to atrial pacing. In the case where the time interval between the pacing pulse and the QRS wave is short, for example, shorter than 0.1 sec., the heartbeat determining section 31 can discriminate the paced beat as that due to ventricular pacing. In the case where a pacing pulse in which the time interval with respect to the QRS wave is long, for example, from 0.1 sec. to 0.2 sec., and that in which the time interval with respect to the QRS wave is short, for example, shorter than 0.1 sec. are consecutively detected immediately before the QRS wave, the heartbeat determining section 31 can discriminate the paced beat as that due to atrial and ventricular pacing.

When the determination result is output from the heartbeat determining section 31 to the tachycardia determining section 32, the tachycardia determining section 32 determines in step S32 whether or not paced beats of the same kind are contained in the heartbeats in a predetermined heart rate or more. For example, the tachycardia determining section 32 determines whether or not paced beats due to atrial pacing, atrial and ventricular pacing, and ventricular pacing are contained in the heartbeats at respective predetermined heart rates or more.

If it is determined that paced beats of the same kind are contained in the heartbeats in the predetermined heart rates or more, the process proceeds to step S33, and the tachycardia determining section 32 determines whether or not the paced beats of the same kind are contained in the heartbeats in a number that is equal to or larger than the predetermined number of consecutive occurrences. For example, the tachycardia determining section 32 determines whether or not paced beats due to atrial pacing, atrial and ventricular pacing, and ventricular pacing occur at respective predetermined numbers of consecutive occurrences or more.

If it is determined that paced beats of the same kind occur at the predetermined number of consecutive occurrences or more, the process proceeds to step S6, and the tachycardia determining section 32 determines that pacing tachycardia occurs in the living body.

As described above, the tachycardia determining section 32 performs the determination for each of paced beats of the same kind, and therefore pacing tachycardia can be determined in detail. In the same or similar manner as Embodiment 2, the tachycardia determining section 32 may preferably determine pacing tachycardia based on the heart rate and number of consecutive occurrences that are set for each kind of paced beats by the user.

If it is determined in step S32 that the number of paced beats of the same kind is smaller than the predetermined heart rate, and in step S33 that the number of paced beats of the same kind is smaller than the predetermined number of consecutive occurrences, the process proceeds to step S8, and the tachycardia determining section 32 determines that pacing tachycardia does not occur in the living body.

According to the embodiment, in the case where the paced beats of the same kind that are discriminated by the heartbeat determining section 31 are in a number that is equal to or larger than the predetermined heart rate, and contained in the heartbeats at the predetermined number of consecutive occurrences or more, the tachycardia determining section 32 determines that pacing tachycardia occurs in the living body, and therefore pacing tachycardia can be determined in detail.

Although, in the embodiment, the tachycardia determining section 32 determines an occurrence of pacing tachycardia based on the number of consecutive occurrences of paced beats, the tachycardia determining section may determine an occurrence of pacing tachycardia based on the occurrence ratio of paced beats in the same or similar manner as Embodiment 2.

### Embodiment 4

In Embodiment 3 described above, in the case where the kind of paced beats that is discriminated by the heartbeat determining section 31 is identical with the kind of paced beats that is previously designated, the tachycardia determining section 32 may preferably determine whether or not paced beats are contained in the heartbeats at the predetermined ratio or more.

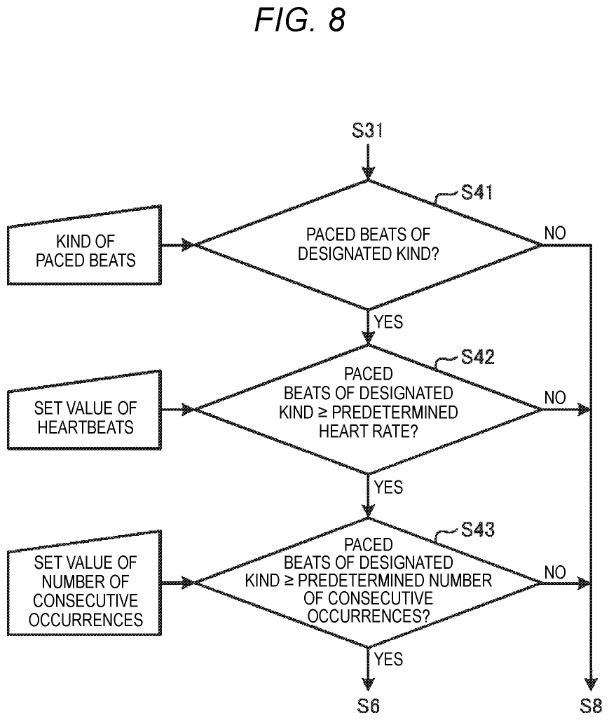

In the same or similar manner as Embodiment 3, as illustrated in FIG. 8, for example, the heartbeat determining section 31 discriminates in step S31 the kind of paced beats, and the result of the discrimination is output from the heartbeat determining section 31 to the tachycardia determining section 32. In the tachycardia determining section 32, here, the kind of paced beats is previously designated by the user. For example, the user may designate one or more of paced beats due to atrial pacing, those due to atrial and ventricular pacing, and those due to ventricular pacing. Therefore, the tachycardia determining section 32 determines in step S41 whether the kind of paced beats that is discriminated by the heartbeat determining section 31 is identical with the kind of paced beats that is previously designated by the user or not.

If it is determined that the kind of paced beats that is discriminated by the heartbeat determining section 31 is identical with the kind of paced beats that is designated by the user, the process proceeds to step S42, and the tachycardia determining section 32 determines whether or not the number of the paced beats that are discriminated by the heartbeat determining section 31 is equal to or larger than a predetermined heart rate or not. If it is determined that the number of the paced beats is equal to or larger than the predetermined heart rate, the process proceeds to step S43, and the tachycardia determining section 32 determines whether the number of the paced beats is equal to or larger than a predetermined number of consecutive occurrences or not. Then, if it is determined that the number of the paced beats is equal to or larger than the predetermined number of consecutive occurrences, the process proceeds to step S6, and the tachycardia determining section 32 determines that pacing tachycardia occurs in the living body.

Then, the notifying section 6 notifies of the kind of paced beats that is discriminated by the heartbeat determining section 31, together with the occurrence of pacing tachycardia.

As described above, the tachycardia determining section 32 determines an occurrence of pacing tachycardia with respect to paced beats of the previously designated kind, and therefore pacing tachycardia can be determined in more detail. Moreover, the notifying section 6 notifies of the kind of paced beats together with an occurrence of pacing tachycardia, and therefore the user can know pacing tachycardia in a more adequate manner.

Preferably, the tachycardia determining section 32 determines pacing tachycardia based on the heart rate and number of consecutive occurrences that are set by the user.

By contrast, if it is determined in step S41 that the paced beats are not of the designated kind, in step S42 that the number of the paced beats of the designated kind is smaller than the predetermined heart rate, and in step S43 that the number of the paced beats of the designated kind is smaller than the predetermined number of consecutive occurrences, the process proceeds to step S8, and the tachycardia determining section 32 determines that pacing tachycardia does not occur in the living body.

According to the embodiment, in the case where the kind of paced beats that is discriminated by the heartbeat determining section 31 is identical with the kind of paced beats that is previously designated, the tachycardia determining section 32 determines an occurrence of pacing tachycardia, and therefore pacing tachycardia can be determined in more detail.

### Embodiment 5

Although, in Embodiments 1 to 4 described above, the tachycardia determining section determines only an occurrence of pacing tachycardia, also an occurrence of spontaneous tachycardia may be determined together with pacing tachycardia.

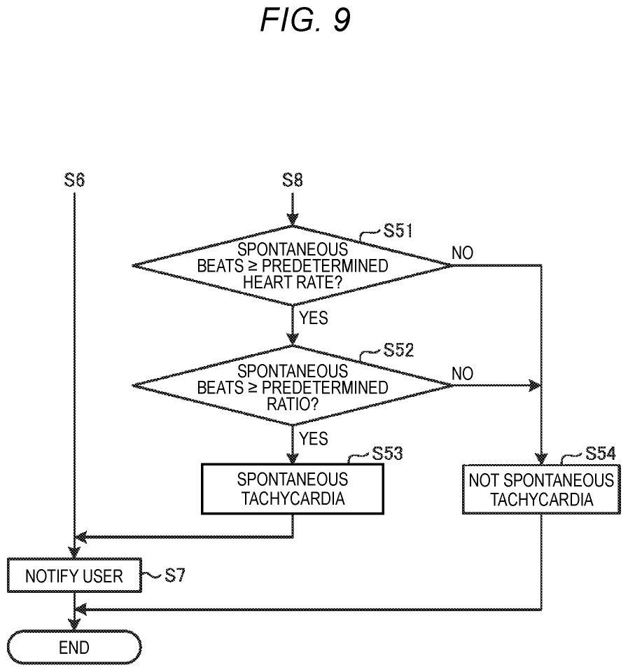

In the same or similar manner as Embodiment 1, as illustrated in FIG. 9, for example, the tachycardia determining section 5 determines in step S8 that pacing tachycardia does not occur. As described in Embodiment 1, the heartbeat determining section 4 determines, in step S3 that is not illustrated, whether the heartbeats detected by the heartbeat detecting section 3a are spontaneous beats of the living body itself or not, based on the detection timings of the pacing pulses that are detected by the pulse detecting section 3b.

Therefore, the tachycardia determining section 5 determines in step S51 whether the number of the spontaneous beats that are determined by the heartbeat determining section 4 is equal to or larger than a predetermined heart rate or not. If it is determined that the number of the spontaneous beats are equal to or larger than the predetermined heart rate, the process proceeds to step S52, and the tachycardia determining section 5 determines whether or not the spontaneous beats are contained in the heartbeats at a predetermined ratio or more.

The heart rate of spontaneous beats indicates the number of the spontaneous beats per minute, and can be obtained based on, for example, the R-R interval of the spontaneous beats, or the number of the spontaneous beats in a constant time period. The ratio of spontaneous beats indicates the generation rate of the spontaneous beats, and, for example, may be obtained from the generated number of spontaneous beats occupying in recent several or several tens of beats, or heartbeats in a constant time period.

Then, if it is determined that the spontaneous beats are contained in the heartbeats at the predetermined ratio or more, the process proceeds to step S53, and the tachycardia determining section 5 determines that spontaneous tachycardia occurs in the living body. The notifying section 6 notifies of, in step S7, an occurrence of spontaneous tachycardia, based on the determination result of the tachycardia determining section 5. In the same or similar manner as Embodiment 1, if the tachycardia determining section 5 determines in step S6 that pacing tachycardia occurs, the notifying section 6 notifies of the occurrence of pacing tachycardia.

As described above, the notifying section 6 notifies of not only an occurrence of pacing tachycardia, but also that of spontaneous tachycardia, and therefore the user can discriminate more clearly between pacing tachycardia and spontaneous tachycardia.

By contrast, if it is determined in step S51 that the number of the spontaneous beats is smaller than the predetermined heart rate, and in step S52 that the spontaneous beats are contained in the heartbeats at a ratio that is smaller than the predetermined ratio, the tachycardia determining section 5 determines in step S54 that spontaneous tachycardia does not occur in the living body, i.e., the spontaneous beats are normal spontaneous beats.

The notifying section 6 may notify that the spontaneous beats are normal spontaneous beats, based on the determination result of the tachycardia determining section 5.

According to the embodiment, in the case where the number of the spontaneous beats is equal to or larger than the predetermined heart rate, and the spontaneous beats are contained in the heartbeats at a ratio that is equal to or larger than the predetermined ratio, the tachycardia determining section 5 determines that spontaneous tachycardia caused by the living body itself occurs, and therefore pacing tachycardia and spontaneous tachycardia can be discriminated more clearly from each other.

In Embodiments 1 to 5 described above, preferably, the tachycardia determining section may further determine whether the difference between a plurality of intervals of the paced beats that are sequentially determined by the heartbeat determining section is within a predetermined range or not, and, if it is determined that the difference between the plurality of intervals of the paced beats is within the predetermined range, the tachycardia determining section determines that pacing tachycardia occurs in the living body. For example, here, the tachycardia determining section may perform the determination based on the difference of the R-R intervals of paced beat. For example, the tachycardia determining section may perform the determination based on: whether the standard deviation of the intervals of paced beats is within a predetermined range or not; whether the difference between the maximum and minimum values of pacing beats is within a predetermined range or not; whether the difference between the intervals of pacing beats and the mean value thereof is within a predetermined range or not; or the like.

According to the configuration, for example, noises can be eliminated, and the tachycardia determining section can determine an occurrence of pacing tachycardia more accurately.

In Embodiments 1 to 5 described above, after the determination of whether or not the number of paced beats is equal to or larger than a predetermined heart rate, the tachycardia determining section determines whether or not the paced beats are contained in the heartbeats at the predetermined ratio or more. However, the tachycardia determining section is requested only to determine whether or not the number of paced beats is equal to or larger than a predetermined heart rate, and whether or not the paced beats are contained in the heartbeats at a predetermined ratio or more, and the manner of the determination is not limited to the above-described manner. For example, after the determination of whether or not paced beats are contained in the heartbeats at the predetermined ratio or more, the tachycardia determining section may determine whether the number of the paced beats is equal to or larger than a predetermined heart rate or not.

According to the presently disclosed subject matter, in the case where the paced beats that are determined by the heartbeat determining section are in a number that is equal to or larger than the predetermined heart rate, and contained in the heartbeats at the predetermined ratio or more, the arrhythmia determining section determines that pacing tachycardia occurs in the living body, and therefore it is possible to provide a heartbeat analyzer that discriminating more clearly pacing tachycardia.

What is claimed is:

1. A heartbeat analyzer that is configured to analyze heartbeats of a living body to which a pacing device for outputting pacing pulses to cause a heart to beat is placed, based on electrical signals of the living body, the heartbeat analyzer comprising:
a heartbeat detecting section that is configured to detect the heartbeats of the living body based on the electrical signals;
a pulse detecting section that is configured to detect outputs of the pacing pulses based on the electrical signals;
a heartbeat determining section that is configured to determine whether the heartbeats detected by the heartbeat detecting section are paced beats due to the pacing pulses detected by the pulse detecting section or not, based on detection timings of the pacing pulses;
a tachycardia determining section that, in a case where the paced beats determined by the heartbeat determining section are in a number that is equal to or larger than a predetermined heart rate, and are contained in the heartbeats at a predetermined ratio or more, is configured to determine that pacing tachycardia caused by the pacing pulses occurs in the living body; and
a notifying section that is configured to notify of the occurrence of the pacing tachycardia based on a result of the determination of the tachycardia determining section.

2. The heartbeat analyzer according to claim 1, wherein, in a case where the paced beats detected by the heartbeat determining section occur in a number that is equal to or larger than a predetermined number of consecutive occurrences, the tachycardia determining section is configured to determine that the paced beats are contained in the heartbeats at the predetermined ratio or more.

3. The heartbeat analyzer according to claim 1, wherein
the heartbeat determining section is configured to discriminate a kind of the paced beats corresponding to a pacing portion due to the pacing pulses, based on detection timings of the heartbeats detected by the heartbeat detecting section, and the detection timings of the pacing pulses detected by the pulse detecting section, and,
in a case where the paced beats of a same kind discriminated by the heartbeat determining section are contained in the heartbeats at the predetermined ratio or more, the tachycardia determining section is configured to determine that the pacing tachycardia occurs.

4. The heartbeat analyzer according to claim 3, wherein, in a case where the kind of the paced beats discriminated by the heartbeat determining section is identical with a kind of a paced beat that is previously designated, the tachycardia determining section is configured to determine whether or not the paced beats are contained in the heartbeats at the predetermined ratio or more.

5. The heartbeat analyzer according to claim 3, wherein the notifying section is configured to notify of the kind of the paced beats discriminated by the heartbeat determining section.

6. The heartbeat analyzer according to claim 1, wherein at least one of the predetermined heart rate and the predetermined ratio is previously set by a user.

7. The heartbeat analyzer according to claim 1, wherein
the heartbeat determining section is configured to determine whether the heartbeats detected by the heartbeat detecting section are spontaneous beats of the living body itself or not, based on the detection timings of the pacing pulses detected by the pulse detecting section,
in a case where the spontaneous beats determined by the heartbeat determining section are in a number that is equal to or larger than a predetermined heart rate, and are contained in the heartbeats at a predetermined ratio or more, the tachycardia determining section is configured to determine that spontaneous tachycardia caused by the living body itself occurs, and
the notifying section is configured to notify of the occurrence of the spontaneous tachycardia based on a result of the determination of the tachycardia determining section.
